(19) United States
(12) Patent Application Publication
(10) Pub. No.: US 2024/0100064 A1
(43) Pub. Date: Mar. 28, 2024

(54) METHODS OF TREATING NEURODEVELOPMENTAL DISORDERS

(71) Applicant: Woolsey Pharmaceuticals, Inc., St. Petersburg, FL (US)

(72) Inventors: Thomas MACALLISTER, Arlington, VA (US); Sven JACOBSON, New York, NY (US)

(21) Appl. No.: 18/516,305

(22) Filed: Nov. 21, 2023

Related U.S. Application Data
(63) Continuation of application No. 17/511,058, filed on Oct. 26, 2021.
Application No. 17/511,058 is a continuation of application No. 17/166,733, filed on Feb. 3, 2021, now abandoned.
(60) Provisional application No. 63/043,859, filed on Jun. 25, 2020.

(51) Int. Cl. A61K 31/551

(52) U.S. Cl. CPC A61K 31/551

(57) ABSTRACT

The invention relates to the treatment of neurodevelopmental disorders using an inhibitor of rho kinase. Preferred methods contemplate the treatment of infants and children. Certain embodiments involve treating a neurodevelopmental disorder is caused by defects of metabolism, including defects of amino acid transport or metabolism, acid-base balance, carbohydrate transport or metabolism, metal homeostasis, neurotransmitter metabolism, or fatty acid transport or metabolism. Methods address a variety of conditions caused by changes in the genetic material that affect the structure and/or expression of certain genes. Preferred methods treat Down syndrome, Fragile X syndrome, oligophrenin 1 deficiency, Rett syndrome, autistic disorder, Asperger's syndrome, pervasive developmental disorder not otherwise specified, and other autism spectrum disorders.

### CROSS-REFERENCE TO RELATED APPLICATIONS

This application is a continuation of U.S. patent application Ser. No. 17/511,058, filed on Oct. 26, 2021, which is a continuation of U.S. patent application Ser. No. 17/166,733, filed on Feb. 3, 2021, which claims priority to U.S. Provisional Application No. 63/043,859, filed on Jun. 25, 2020, the disclosures of each of which are incorporated herein by reference in their entirety.

### BACKGROUND OF THE INVENTION

While resulting in a wide range of phenotypes, ranging from significant behavioral disturbances to cognitive deficits to seizure disorders, neurodevelopmental disorders all result of a failure of the central nervous system (CNS) to properly and fully develop. This leads to abnormal brain function which may affect emotion, learning ability, self-control, motor control and memory. The disorder may occur in response to an active defect present at or before birth or may be due to some triggering event that exposes a predisposition in early life that alters the developmental trajectory or due to a toxic environmental insult.

CNS development is highly regulated and is modulated both by genetic and environmental factors. Perturbations of development early in life can result in missing or abnormal neuronal architecture or connectivity. These perturbations can result from, for example, social deprivation, genetic diseases, metabolic diseases, immune conditions, infectious diseases, poor nutrition, trauma, and exposure to toxins and other environmental factors.

For example, immune reactions during pregnancy, both maternal and of the developing child, that generate immune reactions against the fetal brain tissue may produce neurodevelopmental disorders. These include Pediatric Autoimmune Neuropsychiatric Disorders Associated with Streptococcal infection (PANDAS) and Sydenham's chorea, both of which are due to immune reactions that follow infection by Streptococcus bacteria.

While not considered primary neurodevelopmental disorders, infections can result in neurodevelopmental consequences. Infections that can cause serious neurodevelopmental problems include viral, bacterial, protozoal and parasitic infections and infestations. HIV, for example, may cause meningitis or encephalitis and measles can progress to subacute sclerosing panencephalitis. Other viruses include herpes simplex virus, cytomegalovirus, rubella, and Zika virus. Treponema pallidum may cause congenital syphilis, which may progress to neurosyphilis Plasmodium may result in cerebral malaria and Toxoplasma can cause congenital toxoplasmosis with brain cysts.

A significant cause of neurodevelopmental disorders is an underlying metabolic problem, either in the mother or the child. Diabetes in the child, for example, can produce neurodevelopmental damage by the effects of excess or insufficient glucose. Gestational diabetes in the mother can cause similar issues. Phenylketonuria either in the fetus or the mother, resulting in excessive phenylalanine, can cause intellectual disability.

Nutrition disorders and nutritional deficits may cause neurodevelopmental disorders, such as those resulting from neural tube defects, like spina bifida or anencephaly. Maternal folic acid deficiency is the most common nutritional cause of neural tube defects, but may also be caused by genetic modifications, medications or other environmental causes that interfere with folate metabolism. Iodine deficiency also produces neurodevelopmental disorders resulting in intellectual disability. Other dietary causes include alcohol, resulting in fetal alcohol syndrome and iron supplementation in the newborn has been linked to delayed neurodevelopment.

Brain trauma during development is a common cause of neurodevelopmental syndromes. It may be congenital, often due to asphyxia, hypoxia or mechanical trauma from birth, or a result of injury in infancy or childhood.

Neurodevelopmental disorders include a broad spectrum of disorders referred to by the DSM-5 as intellectual disability (formerly known as mental retardation). Examples of such conditions include Down syndrome, Fragile X syndrome, fetal alcohol syndrome and a number of metabolic disorders. Autism spectrum disorder is another example of a commonly known neurodevelopmental disorder.

Many neurodevelopmental disorders have a genetic basis and may be confirmed by genetic testing based on defects in certain genes or gross chromosomal aberrations known to be associated with conditions manifesting the observed symptomatology. Often this is done by chromosomal microarray analysis because it can detect a wide range of chromosome abnormalities and duplications or deletions (copy-number variants) involving the coding or regulatory regions associated with specific genes.

Effective treatments do not exist for many neurodevelopmental conditions, with treatment generally related to addressing specific symptoms, but not the underlying condition. In some cases, especially for certain metabolic conditions, diet many be used to manage the patient, alleviating symptoms and slowing the damage. It is the hope that some of the genetically-based conditions may be addressed by newer technologies that can replace or repair the defective gene or supplement it using RNA that can at least transiently provide a clean copy via which missing or defective genes may be expressed. There is a need for new agents that can intervene in broad neurodevelopmental pathways that are interrupted in neurodevelopmental conditions in order to arrest or reverse the underlying damage.

### SUMMARY OF THE INVENTION

The invention relates to methods of treating a neurodevelopmental disorders using a rho kinase inhibitor. Preferred methods contemplate treating patients under 18 years old with some preferred methods treating patients under 18 months old.

In one embodiment the methods are achieved by administering a rho kinase inhibitor in a dose of 1 to 2 mg per kilogram of body weight per day. Fasudil is a preferred rho kinase inhibitor and preferred methods utilize oral administration.

A preferred embodiment involves treating a neurodevelopmental disorder that is caused by a metabolic perturbation. These may be, for example, caused by defects of amino acid transport or metabolism, acid-base balance, carbohydrate transport or metabolism, metal homeostasis, neurotransmitter metabolism, or lipid or fatty acid transport or metabolism.

Disorders treatable according to the invention may have a genetic and/or an environmental basis. Some inventive methods involve treating a condition that results from a deletion or duplication of genetic material. Such genetic alterations may be, for example, in the coding and/or regulatory regions of the genetic material.

In some embodiments, the disorder is selected from the group consisting of Down syndrome, Fragile X syndrome, oligophrenin 1 deficiency, Rett syndrome, autistic disorder, Asperger's syndrome, pervasive developmental disorder not otherwise specified.

### DETAILED DESCRIPTION OF THE INVENTION

The invention relates to the discovery that rho kinases play a central role in regulating neural development and that they are induced in a number of pathological processes. The use of rho kinase inhibitors, therefore, may be employed to beneficial effect to treat a variety of these disorders, whether caused my genetic defect, trauma or some other environmental factor. Specific contemplated disorders are described below.

### ROCK Inhibitors

The inventive methods contemplate the administration of a rho kinase (ROCK) inhibitor in the treatment of a disease or condition. Two mammalian ROCK homologs are known, ROCK1 (aka ROKβ, Rho-kinase β, or p160ROCK) and ROCK2 (aka ROKα) (Nakagawa 1996). In humans, the genes for both ROCK1 and ROCK2 are located on chromosome 18. The two ROCK isoforms share 64% identity in their primary amino acid sequence, whereas the homology in the kinase domain is even higher (92%) (Jacobs 2006; Yamaguchi 2006). Both ROCK isoforms are serine/threonine kinases and have a similar structure.

A large number of pharmacological ROCK inhibitors are known (Feng, LoGrasso, Defert, & Li, 2015). Isoquinoline derivatives are a preferred class of ROCK inhibitors. The isoquinoline derivative fasudil was the first small molecule ROCK inhibitor developed by Asahi Chemical Industry (Tokyo, Japan). The characteristic chemical structure of fasudil consists of an isoquinoline ring, connected via a sulphonyl group to a homopiperazine ring. Fasudil is a potent inhibitor of both ROCK isoforms. In vivo, fasudil is subjected to hepatic metabolism to its active metabolite hydroxyfasudil (aka, M3). Other examples of isoquinoline derived ROCK inhibitors include dimethylfasudil and ripasudil.

Other preferred ROCK inhibitors are based on based on 4-aminopyridine structures. These were first developed by Yoshitomi Pharmaceutical (Uehata et al., 1997) and are exemplified by Y-27632. Still other preferred ROCK inhibitors include indazole, pyrimidine, pyrrolopyridine, pyrazole, benzimidazole, benzothiazole, benzathiophene, benzamide, aminofurazane, quinazoline, and boron derivatives (Feng et al., 2015). Some exemplary ROCK inhibitors are shown below:

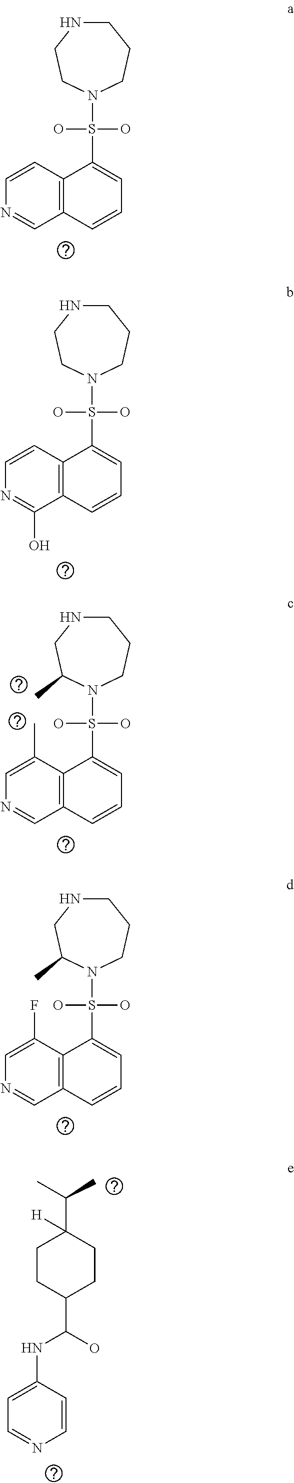

ROCK inhibitors according to the invention may have more selective activity for either ROCK1 or ROCK2 and will usually have varying levels of activity on PKA, PKG, PKC, and MLCK. Some ROCK inhibitors may be highly specific for ROCK1 and/or ROCK2 and have much lower activity against PKA, PKG, PKC, and MLCK.

A particularly preferred ROCK inhibitor is fasudil. Fasudil may be exist as a free base or salt and may be in the form of a hydrate, such as a hemihydrate. As used herein, unless specifically noted, the name of any active moiety, such as fasudil, should be considered to include all forms of the active moiety, including the free acid or base, salts, hydrates, polymorphs and prodrugs of the active moiety.

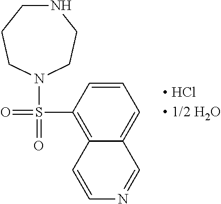 [SMILES: O=S(=O)(c1cccc2cnccc12)N1CCCNCC1]

### Hexahydro-1-(5-isoquinolinesulfonyl)-1H-1,4-diazepine monohydrochloride hemihydrate

Fasudil is a selective inhibitor of protein kinases, such as ROCK, PKC and MLCK and treatment results in a potent relaxation of vascular smooth muscle, resulting in enhanced blood flow (Shibuya 2001). A particularly important mediator of vasospasm, ROCK induces vasoconstriction by phosphorylating the myosin-binding subunit of myosin light chain (MLC) phosphatase, thus decreasing MLC phosphatase activity and enhancing vascular smooth muscle contraction. Moreover, there is evidence that fasudil increases endothelial nitric oxide synthase (eNOS) expression by stabilizing eNOS mRNA, which contributes to an increase in the level of the potent vasodilator nitric oxide (NO), thereby enhancing vasodilation (Chen 2013).

Fasudil has a short half-life of about 25 minutes, but it is substantially converted in vivo to its 1-hydroxy (M3) metabolite. M3 has similar effects to its fasudil parent molecule, with slightly enhanced activity and a half-life of about 8 hours (Shibuya 2001). Thus, M3 is likely responsible for the bulk of the in vivo pharmacological activity of the molecule. M3 exists as two tautomers, depicted below:

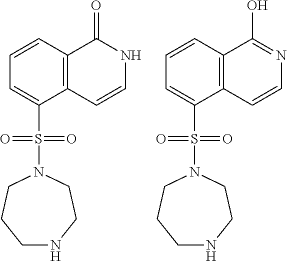

The ROCK inhibitors used in the invention, such as fasudil, include pharmaceutically acceptable salts and hydrates. Salts that may be formed via reaction with inorganic and organic acid. Those inorganic and organic acids are included as following: hydrochloric acid, hydrobromide acid, hydriodic acid, sulphuric acid, nitric acid, phosphoric acid, acetic acid, maleic acid, maleic acid, maleic acid, oxalic acid, oxalic acid, tartaric acid, malic acid, mandelic acid, trifluoroacetic acid, pantothenic acid, methane sulfonic acid, or para-toluenesulfonic acid.

### Pharmaceutical Compositions

Pharmaceutical compositions of ROCK inhibitors usable in the are generally oral and may be in the form of tablets or capsules and may be immediate-release formulations (i.e., those in which no elements of the formulation are designed to substantially control or retard the release of the ROCK inhibitor upon administration) or may be controlled- or extended-release formulations, which may contain pharmaceutically acceptable excipients, such as corn starch, mannitol, povidone, magnesium stearate, talc, cellulose, methylcellulose, carboxymethylcellulose and similar substances. A pharmaceutical composition comprising a ROCK inhibitor and/or a salt thereof may comprise one or more pharmaceutically acceptable excipients, which are known in the art. Formulations include oral films, orally disintegrating tablets, effervescent tablets and granules or beads that can be sprinkled on food or mixed with liquid as a slurry or poured directly into the mouth to be washed down.

Pharmaceutical compositions containing ROCK inhibitors, salts and hydrates thereof can be prepared by any method known in the art of pharmaceutics. In general, such preparatory methods include the steps of bringing a ROCK inhibitor or a pharmaceutically acceptable salt thereof into association with a carrier or excipient, and/or one or more other accessory ingredients, and then, if necessary and/or desirable, shaping, and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. The composition used in accordance with the methods of the present invention may comprise between 0.001% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

In certain embodiments, the pharmaceutical composition used in the methods of the present invention may comprise a diluent. Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

In certain embodiments, the pharmaceutical composition used in the methods of the present invention may comprise a granulating and/or dispersing agent. Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (VEEGUM), sodium lauryl sulfate, quaternary ammonium compounds, and mixtures thereof.

In certain embodiments, the pharmaceutical composition used in the methods of the present invention may comprise a binding agent. Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (VEEGUM®), and larch arabogalactan), alginates, polyethylene oxide, polyethylene glycol, inorganic calcium salts, silicic acid, polymethacrylates, waxes, water, alcohol, and/or mixtures thereof.

In certain embodiments, the pharmaceutical composition used in the methods of the present invention may comprise a preservative. Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

In certain embodiments, the pharmaceutical composition used in the methods of the present invention may comprise an antioxidant. Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

In certain embodiments, the pharmaceutical composition used in the methods of the present invention may comprise a chelating agent. Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

In certain embodiments, the pharmaceutical composition may comprise a buffering agent together with the ROCK inhibitor or the salt thereof. Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

In certain embodiments, the pharmaceutical composition used in the methods of the present invention may comprise a lubricating agent. Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

In other embodiments, the pharmaceutical composition of containing a ROCK inhibitor or salt thereof will be administered as a liquid dosage form. Liquid dosage forms for oral and parenteral administration include pharmaceutically acceptable emulsions, microemulsions, solutions, suspensions, syrups, and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates of the invention are mixed with solubilizing agents such as Cremophor™, alcohols, oils, modified oils, glycols, polysorbates, cyclodextrins, polymers, and mixtures thereof.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Some compositions of the invention relate to extended- or controlled-release formulations. These may be, for example, diffusion-controlled products, dissolution-controlled products, erosion products, osmotic pump systems or ionic resin systems. Diffusion-controlled products comprise a water-insoluble polymer which controls the flow of water and the subsequent egress of dissolved drug from the dosage from. Dissolution-controlled products control the rate of dissolution of the drug by using a polymer that slowly solubilizes or by microencapsulation of the drug—using varying thicknesses to control release. Erosion products control release of drug by the erosion rate of a carrier matrix. Osmotic pump systems release a drug based on the constant inflow of water across a semi permeable membrane into a reservoir which contains an osmotic agent. Ion exchange resins can be used to bind drugs such that, when ingested, the release of drug is determined by the ionic environment within the gastrointestinal tract.

### Treatable Patients

Patients treatable according to the invention are those with a known or suspected neurodevelopmental disorder. As used herein, a neurodevelopmental disorder is defined as any condition that results in a failure of the CNS to properly and fully develop, irrespective of underlying pathology or etiology. This disorder may occur as a result of an active defect (genetic or otherwise) present at or before birth or may be due to some event early life that alters the developmental trajectory or due to a toxic environmental insult. It may be due to genetic and/or environmental factors. Such conditions lead to abnormal brain function which may affect emotion, learning ability, self-control, motor control and memory. Examples of neurodevelopmental disorders that may be treated according to the invention are discussed below.

Neurodevelopmental disorders include those of autoimmune origin. These include Pediatric Autoimmune Neuropsychiatric Disorders Associated with Streptococcal infection (PANDAS) and Sydenham's chorea, both of which are due to immune reactions that follow infection by Streptococcus bacteria. In such conditions, the body's immune response is misdirected against the patient's brain tissues.

In addition to inducing autoimmune disorders, viral, bacterial, protozoal and parasitic infections and infestations can cause serious neurodevelopmental problems via other mechanisms. Human immunodeficiency virus, for example, may cause meningitis or encephalitis and measles can progress to subacute sclerosing panencephalitis. Other viral causes include herpes simplex virus, cytomegalovirus, rubella, and Zika virus. Treponema pallidum may cause congenital syphilis, which may progress to neurosyphilis, Plasmodium may result in cerebral malaria and Toxoplasma can cause congenital toxoplasmosis with brain cysts.

One of the most significant causes of neurodevelopmental disorders is an underlying metabolic problem, either in the mother or the child. Commonly, these may affect amino acids, acid-base balance, carbohydrate metabolism, elevated or reduced levels of metals, neurotransmitter metabolism, and/or fatty acid transport and metabolism.

Because the brain is dependent on carbohydrate metabolism (primarily glucose and secondarily lactate), defects affecting these can have profound effects on neurodevelopment. Such disorders of energy utilization may implicate carbohydrate transport and/or metabolism and include galactosemias of galactose, galactose-1-phosphate and galactitol. Glucose transporter 1 (GLUT 1) deficiency can cause low glucose levels in the cerebrospinal fluid, resulting in abnormal energy utilization. Defects in the monocarboxylic acid transporter (SLC16A1) can result in perturbed energy utilization of lactate, pyruvate and ketone bodies. Other perturbations of energy production are also implicated. Defects in the aspartate-glutamate carrier from brain mitochondria (ARALAR/AGC1/S1c25a12), a key player in maintaining oxidative glucose utilization, can also cause neurodevelopmental disorders and is characterized by reduced levels of N-acetyl aspartate and elevated levels of lactate in the cerebrospinal fluid. Diabetes is another example of disrupted carbohydrate metabolism that may have profound neurodevelopmental effects. Diabetes in the child, for example, can produce neurodevelopmental damage by the effects of excess or insufficient glucose. Gestational diabetes in the mother can cause similar issues.

Amino acid defects are also known to cause neurodevelopmental disorders. These include disorders of amino acid catabolism, like phenylketonuria, homocysteinurias, maple syrup urine disease and urea cycle disorders. Phenylketonuria either in the fetus or the mother is a well-known example that results in excessive phenylalanine and can cause intellectual disability. Neurodevelopmental disorders can also include deficiencies in other amino acid levels. Specifically, they include: branched chain amino acid deficiencies caused by defects in the large neutral amino acid transporter (SLC7A5) and/or the branched chain dehydrogenase kinase; glutamine deficiency caused by defective glutamine synthetase; low serine levels caused by defects in 3-phosphoglycerate dehydrogenase, phosphoserine aminotransferase, phosphoserine phosphatase, or the serine (SL-C1A4) transporter; and asparagine deficiency caused by defective asparagine synthetase. Defects in serine metabolism can cause Neu-Laxova syndrome.

Organic acidurias may cause neurodevelopmental disorders, including propionic, methylmalonic, and isovaleric acidurias; cerebral organic acidurias like glutaric aciduria type 1; other cerebral organic acidurias, like L2 hydroxyglutaric, D2 hydroxyglutaric, DL hydroxyglutaric aciduria and Canavan disease (N-acetylaspartate aciduria).

Imbalances in metals cause many neurodevelopmental disorders. These include disorders of metal accumulation, including copper, iron and manganese. Copper accumulation may occur due to ATP7B transporter defects; iron accumulation may be due to pantothenate kinase, coenzyme A synthetase, PLA2G6, C19oef12, FAH2, WDR45, ATP13A2, FTL, DCAF17, SCP2 or GTPBP2 defects; and manganese due to defects in the SLC30A10 and/or SLC30A14 transporters. Low serum copper (and its transporter ceruleoplasmin) can be caused by deficits in Menkes' protein (ATP7A) or ANSI the latter causing MEDNIK syndrome (mental retardation, enteropathy, deafness, neuropathy, ichthyosis, keratodermia), characterized by abnormal copper trafficking; and low manganese and zinc levels by defects in SLC39A8.

Neurotransmitter defects are another class of metabolic disturbances that can cause neurodevelopmental disorders. These include monoamine synthesis defects as a result of defective tyrosine hydroxylase, aromatic amino acid decarboxylase, DNA-JC12, tetrahydrobiopterin deficiencies, deficiencies in guanosine triphosphate cyclohydroxylase, sepiapterine reductase, dihydropterine reductase, or 6-pyruvoyl-tetrahydropterin synthase. They also include monoamine transport defects affecting the dopamine transporter or brain vesicular monoamine transporter 2. Gamma amino butyric acid defects resulting from succinyl adenosine dehydrogenase or glutamate and/or glycine receptors and/or transporters are also included, as are glycine defects/non-ketotic hyperglycinemias resulting from glycine cleavage system and lipoate disorders.

Defects in fatty acid transport and/or metabolism can also be the cause of neurodevelopmental disorders. One example is a defect in Mfsd2a, a transporter for the essential omega-3 fatty acid docosahexaenoic acid (DHA); it transports DHA in the form of lysophosphatidylcholine. Alterations of this gene cause elevated plasma lysophosphatidylcholine and reduced brain levels of DHA, one of the major structural fatty acids in the brain and necessary for development. Defects in fatty acid metabolism include defects in fatty acid elongation factors, like ELOVL4 and ELOVL5 (resulting in low serum C20-4 and C22-6) or fatty aldehyde dehydrogenase (FALDH; ALDH3A2), which functions to remove toxic aldehydes that are generated by lipid peroxidation. Mutations in the PLA2G6 gene have been linked to infantile neuroaxonal dystrophy. The PLA2G6 encodes an A2 phospholipase, which is involved in breaking metabolizing phospholipids.

Defects of lipid storage may be the cause of neurodevelopmental disorders. Lipid storage diseases include Gaucher disease, Niemann-Pick disease, Fabry disease, Farber's disease, gangliosidoses, Krabbe disease, Metachromatic leukodystrophy, and Wolman's disease. Gaucher disease is caused by a deficiency of the enzyme glucocerebrosidase. Specifically, Type 2 Gaucher (acute infantile neuropathic Gaucher disease) is characterized by extensive and progressive brain damage with neuromuscular symptoms like spasticity, seizures, limb rigidity, abnormal eye movement, and a poor ability to suck and swallow. Niemann-Pick disease is a group of autosomal recessive disorders caused by an abnormal accumulation of fat and cholesterol. Neurological complications may include ataxia, eye paralysis, brain degeneration, learning problems, spasticity, feeding and swallowing difficulties, slurred speech, and loss of muscle tone. Types A and B disease are due to accumulation of sphingomyelin, due to defective sphingomyelinase. Type C is caused by a lack of the NPC1 or NPC2 proteins, which notably causes cholesterol to accumulate inside nerve cells.

Fabry disease is an X-linked alpha-galactosidase-A deficiency that causes a buildup of fatty material in the autonomic nervous system. Neurological signs include burning pain in the arms and legs. Fatty storage in blood vessel walls may impair circulation, putting the person at risk for stroke. Farber's disease, also known as Farber's lipogranulomatosis, is a group of rare autosomal recessive disorders caused by a deficiency in ceramidase, resulting in accumulation of fatty material in the central nervous system. Neurological symptoms usually develop within the first few weeks of life that may include increased lethargy and sleepiness, and problems with swallowing and breathing. The gangliosidoses are comprised of two groups of autosomal recessive genetic conditions: the GM1 and GM2 subtypes. The GM1 subtype is caused by a deficiency of the enzyme beta-galactosidase, resulting in abnormal storage of acidic lipid materials in nerve cells. The GM2 subtype results from a deficiency of beta-hexosaminidase and also causes the body to store excess acidic fatty materials in in nerve cells. Tay-Sachs disease (also known as GM2 gangliosidosis-variant B) and is caused by a deficiency in the enzyme hexosaminidase A. Sandhoff disease (variant AB) is a severe form of Tay-Sachs disease.

Krabbe disease (also known as globoid cell leukodystrophy and galactosylceramide lipidosis) is an autosomal recessive disorder caused by deficiency of the enzyme galactocerebrosidase. The buildup of fats affects the development of the myelin sheath and causes severe deterioration of mental and motor skills. Metachromatic leukodystrophy, or MLD, is a group of autosomal recessive disorders that affect the myelin sheath that is characterized by buildup in the white matter of the central nervous system and in the peripheral nerves. Wolman's disease, also known as acid lipase deficiency, is an autosomal recessive disorder resulgin in the accumulation of cholesteryl esters and triglycerides, leading to progressive mental deterioration.

Neurodevelopmental disorders may be caused by vitamin and/or cofactor defects. Biotin deficiencies, for example, may be due to biotinidase defects. Cobalamin C (Cbl-C) defect causes impaired conversion of dietary vitamin B12 into its two metabolically active forms, methylcobalamin and adenosylcobalamin. Methylene tetrahydrofolate reductase defects causing problems of folic acid metabolism that result in neural tube defects. Errors of vitamin B6 (pyridoxine) metabolism result from deficiencies in antiquitin (ALDH7A1; α-aminoadipic semialdehyde dehydrogenase) or pyridox(am)ine 5′-phosphate oxidase (PNPO). Thiamine pyrophosphate depletion can result from defects in the mitochondrial thiamine pyrophosphate transporter (SLC25A19), the thiamine transporter (SLC19A3) or thiamine pyrophosphate kinase.

Nutrition disorders and nutritional deficits may cause neurodevelopmental disorders, such as the neural tube defects spina bifida or anencephaly. Maternal folic acid deficiency is the most common nutritional cause of neural tube defects, but may also be caused by genetic modifications (as set out above), medications or other environmental causes that interfere with folate metabolism. Iodine deficiency also produces neurodevelopmental disorders resulting in intellectual disability. Other dietary causes include alcohol, resulting in fetal alcohol syndrome and iron supplementation in the newborn has been linked to delayed neurodevelopment.

Brain trauma during development is a common cause of neurodevelopmental syndromes. It may be congenital, often due to asphyxia, hypoxia or mechanical trauma from the birth process, or a result of injury in infancy or childhood.

Perhaps the most well-known neurodevelopmental disorder, Autism spectrum disorder (ASD) is a disorder that affects communication and behavior. Known as a “spectrum” disorder, autism exhibits a wide variation in the type and severity of symptoms people experience. According to the Diagnostic and Statistical Manual of Mental Disorders (DSM-5), people with ASD have difficulty with communication and interaction with other people, restricted interests and repetitive behaviors, and symptoms that adversely affect the ability to function properly in daily life. Prior to 2013, the DSM separately classified autistic disorder, Asperger's' syndrome and pervasive developmental disorder not otherwise specified (PDD-NOS) as separate conditions, but they are now properly considered to be part of ASD.

Individuals with ASD are reported to have atypical white matter developmental patterns compared to those without ASD. Changes in white matter volume and connectivity are measured by diffusion tensor imaging (DTI), which is a form of MRI and measures the diffusion of water molecules throughout the brain. Dysfunctional longer white matter tracts, for example between the frontal lobes and basal ganglia, or cerebellum, basal ganglia, and neocortex, may contribute to narrow repetitive behaviors. Decreased functional connectivity between cerebellar and cortical regions has also been observed. However it is unclear whether these are specific to autism or are consequences of associated disorders such as epilepsy.

There are no accepted biomarkers for ASD, although deletions of neurexin 1 (NRXN1), mutations of SHANK3 and SHANK2 and duplications at 7q11.23, 16p11.2, and 15q11-13 have been reported.

The exact causes of ASD are unknown, but research suggests it is due to a combination of genetics with influences from the environment that affect development. Significant risk factors include having a sibling with ASD, having older parents, having certain genetic conditions like Down syndrome, fragile X syndrome, and Rett syndrome, and very low birth weight.

Often also associated with ASD, and an early example of a copy number variate disorder, the most common genetic chromosomal disorder and cause of learning disabilities in children, Down syndrome is caused when abnormal cell division results in an extra full or partial copy of chromosome 21. There are three genetic variations that can cause Down syndrome:

Trisomy 21 comprises about 95 percent of cases and these individuals have three copies of chromosome 21 in all the cells of the body, rather than the normal two. The extra chromosome results from disjunction during the development of the sperm cell or the egg cell.

Mosaic Down syndrome is a rare form of the disease where only some cells of the body have an extra copy of chromosome 21. This is caused by disjunction during cell division after fertilization.

Translocation Down syndrome occurs when a portion of chromosome 21 becomes attached (translocated) onto another chromosome. These individuals have the normal two copies of chromosome 21, along with additional genetic material from chromosome 21 attached to another chromosome.

In all three variants, this extra genetic material causes the developmental changes and physical features of Down syndrome. While it can vary in severity, Down syndrome causes lifelong intellectual disability and developmental delays and commonly causes other medical conditions, including heart and gastrointestinal disorders. Most children with Down syndrome have mild to moderate cognitive impairment, manifesting mainly as delayed language and impaired short and long-term memory.

Fragile X syndrome (FXS) is the most common inherited form of intellectual disability that is associated with autism. FXS is an X-linked monogenic disorder resulting from a loss of function of the fragile X mental retardation protein (FMRP). FMRP, encoded by the FMR1 gene, is an RNA binding protein abundantly expressed in the brain. FMRP interacts with messenger RNAs that encode pre- and post-synaptic proteins that are important for plasticity. It appears to control their local translation by controlling ribosomal speed. In most patients, expansion of the CGG trinucleotide repeats—targets of methylation—in the upstream control region of the FMR1 gene results in widespread methylation and transcriptional silencing of the gene and “immature” spine morphologies commonly observed early in development persist in adults. Protein translation defects resulting from the loss of FMRP function are thought to disrupt synaptic maturation and plasticity, thereby affecting neuronal development and repair.

Rett syndrome (RTT) is a progressive neurological disorder that primarily affects girls, occurring in 1 in 10,000-15,000 live female births. Following a period of normal neurological and physical development during the up to the first year-and-a-half of life and appear progressively over several stages of progression, stability, regression and deterioration, potentially throughout life. Symptoms of RTT include loss of acquired speech and motor skills, repetitive hand movements, breathing irregularities and seizures. RTT patients may also suffer from sporadic episodes of gastrointestinal problems, hypoplasia, early-onset osteoporosis, bruxism and screaming spells. Originally thought to be a metabolic condition, RTT patients often present with dyslipidemia, elevated levels of leptin, adiponectin, and ammonia. Energy production also is affected, with reports of abnormal brain carbohydrate metabolism and mitochondrial structure, along with altered electron transport chain complex function, increased oxidative stress, and elevated levels of lactate and pyruvate in blood and cerebrospinal fluid. Mutations in the oligophrenin 1 gene on Xq12 is one of the X-linked genes responsible for impaired mental development. In addition to mental retardation, these patients display clinical features like epilepsy, fronto-temporally pronounced ventriculomegaly, cerebellar hypoplasia and in part strabismus.

Along with Down syndrome, so called copy number variation (CNV) disorders arise from the dosage imbalance of one or more genes, resulting from deletions, duplications or other genomic rearrangements that lead to the loss or gain of genetic material.

The most common recurrent CNV disorder is 22q11.2 deletion syndrome (formerly DiGeorge or velocardiofacial syndrome). 22q11.2 deletion may cause problems with development and function of the brain, resulting in learning, social, developmental or behavioral problems, with some developing attention-deficit/hyperactivity disorder or autism spectrum disorder. Prader-Willi syndrome, caused by deletion of a part of chromosome 15 (15q11-q13) inherited from the father, can cause mild to moderate intellectual disability. Angelman syndrome, also caused by a deletion in 15q11-q13 that affects the ubiquitin protein ligase E3A (UBE3A) gene, results in intellectual disability. Williams-Beuren syndrome (WBS; Williams syndrome) also causes intellectual disability. It results from a deletion in 7q11.23 that may affect up to 28 different genes including the ELN (elastin) gene, the LIMK1 (or LIM kinase-1) gene, and the RFC2 (replication factor C, subunit 2) gene.

Microarray-based CNV analysis increasingly has identified genomic disorders and syndromes that have been directly associated with deletion and/or duplication of genetic material. Many new microdeletion and microduplication syndromes are associated with 1q21.1, 16p11.2 and 15q13.3. Others identified include: 2q11.2; 2q13; 7q36.1; 8p23.1; 10q11.21-q11.23; 10q22-q23; 15q24; 16p11.2p12.2; 16p12.1; 16p13.11; 17q12; 17821.31; Distal 22811.23; and Xp11.22-p11.23.

Other neurodevelopmental disorders treatable according to the invention include: PLAA-(phospholipase A2) associated neurodevelopmental disorder; Neurodevelopmental disorder with severe motor impairment and absent language (NEDMIAL); GRIN1-associated disorders

PURA syndrome; Deformed epidermal autoregulatory factor-1 (DEAF1)-associated disorders; Micro syndrome; Stankiewicz-Isidor syndrome; CHD2 (chromodomain helicase DNA binding protein 2) myoclonic encephalopathy; SCN2A related disorders; Bain type of X-linked syndromic intellectual disability; IRF2BPL (interferon regulatory factor 2 binding protein-like)-related disorders; Smith-Kingsmore syndrome; Chromosome 15q25.2 microdeletion; GNAO1 (G Protein Subunit Alpha 01) encephalopathy; Cerebrooculonasal syndrome; 2q23.1 microdeletion syndrome; SETBP1 disorder; Ethylmalonic encephalopathy; Phosphoribosylpyrophosphate synthetase superactivity; Potocki-Lupski syndrome; Pierson syndrome; Atypical Rett syndrome (CDKL5 deficiency); Multiple congenital anomalies-hypotonia-seizures syndrome type 2; Ornithine translocase deficiency syndrome; Dihydrolipoamide dehydrogenase deficiency; Tuberous sclerosis complex; and Beckwith-Wiedemann syndrome

Animal models exist for some of the disorder above, including ASD and Fragile X, but a grave degree of skepticism should be applied in interpreting animal data. Even aside from the obvious issues of differences in brain complexity between rodents and humans, many of the existing models bear only a passing resemblance to the human condition. Many things can cause neural developmental disorder in animals and many putative drugs can show positive effects in animal models but not in humans. It is crucial, therefore, that animal models, with their known deficiencies in the best of cases, as closely resemble the human disease as possible, in both pathology and clinical presentation. To date, no such models exist for most neurodevelopmental disorders. Moreover, successful pharmacologic intervention in animal models typically does not translate to humans. As one example, a mouse model for Fragile X syndrome has been available for more than 20 years, but all attempts to replicate pre-clinical findings in human trials have failed. (Dahlhaus 2018). Dahlhaus observes that “[i]n the FXS field for instance, more than 70 studies reporting rescues (excluding reviews) have been published on pubmed during the last 12 years, 63 clinical trials are registered on ClinicalTrials.gov, and not a single treatment is available for patients yet.”

Fasudil has been administered to oligophrenin-1-deficient mice, which display overactivation of ROCK and protein kinas A signaling (Allegra et al. 2017). Fasudil was not effective in rescuing axon formation but restored spine density. Fasudil treatment also has been shown to rescue hippocampal hyperexcitability and reverse behavioral changes and brain ventricular enlargement in oligophrenin-1 deficient mice. (Busti 2020) (Meziane et al. 2016).

### Learning Disabilities

The patients treatable according to the invention will generally have intellectual disability (ID; formerly known a mental retardation) is usually defined as an overall IQ of <70. ID can be roughly classified into syndromic ID and non-syndromic ID. Generally, syndromic ID is clinically recognizable due to a specific pattern of physical, neurological, (neuro)radiological or metabolic features combined with ID. Individuals whose only consistent clinical manifestations are impaired cognitive functions are designated as non-syndromic ID. Patients with either syndromic and non-syndromic ID may be treated.

Because the most effective interventions for neurodevelopment will be deployed early in life, most patients treated according to the invention will not yet be adults and generally will be younger than 18 or even 16 years old. More preferably, patients will be under 14 or even under 12 years old. Even more preferably, the patients will be infants or children. Most preferably, patient will be under the age of 36 months in order to obtain maximum effectiveness.

Patients may be identified at any age, but younger patients are generally identified by using one or more neurodevelopmental scales that are used to determine the patient's developmental status according to a norm. Various scales have been employed and used to detect neurodevelopmental deficits and these may be used to assess both the appropriate patient for intervention and the results of the inventive methods.

The Hammersmith Neonatal Neurological Examination (HNNE) encompasses 34 items assessing tone, motor patterns, observation of spontaneous movements, reflexes, visual and auditory attention and behavior.

The Hammersmith Infant Neurological Examination (HINE) is based on the same principles as the HNNE and consists of 26 items that assess different aspects of neurological function: cranial nerve function, movements, reflexes and protective reactions and behavior, as well as some age-dependent items that reflect the development of gross and fine motor function. The HINE is used for infants between 3 and 24 months of age. The HINE is quick to perform and often used.

Considered the “gold standard,” The Bayley Scales of Infant and Toddler Development is used primarily to assess the development of infants and toddlers, ages 1-42 months. It derives a developmental quotient (DQ) rather than an intelligence quotient (IQ). Scores are used to determine the child's performance compared with norms. It contains 5 subscales: Cognitive (aka Mental Development Index), Language, Motor, Social-Emotional, and Adaptive Behavior. It must be administered by a trained assessor.

The Ages and Stages Questionnaire (ASQ; currently ASQ-3)) is also widely used and has the advantage that it is a parent-completed screening tool. It measures developmental milestones in five domains: communication, fine motor, gross motor, problem solving ability, and personal-social functioning. Each domain consists of six questions. Parents indicate whether their child has mastered the milestone (yes, 10 points), partly/inconsistently (partly, 5 points), or not yet (no, 0 points).

The Standardized Infant NeuroDevelopmental Assessment (SINDA) neurological scale is applicable in the age range of 6 weeks to 12 months corrected age (true age minus the number of weeks the child is premature) and results in a score that is largely independent of the infant's age. It has five domains assessing spontaneous movements (eight items), cranial nerve function (seven items), motor reactions (five items), muscle tone (four items), and reflexes (four items).

Picciolini (2005) also published a widely used neurofunctional assessment tool that has been used in infants with low birth weight. The Touwen Infant Neurological Examination (TINE is also used to assess neurological dysfunction in infants (Touwen B C L. Neurological development in infancy. Clinics in Dev. Med. 1976; 58. London: Mac Keith Press).

The Amiel-Tison Neurological Assessment at Term (ATNAT) consists of three different instruments that share the same methodology and a similar scoring system, but are adapted for us in children from 32 weeks post-conception to 6 years of age. The ATNAT takes 5 minutes to administer and has been used in clinical and research settings.

For ASD, specifically, changes from baseline can be evaluated and even distinguished from other neurodevelopmental disorders using the Baby and Infant Screen for Children with aUtIsm (BISCUIT). (Matson 2009). The first component is an informant-based measure designed to assess symptoms of Autistic disorder and pervasive developmental disorder not otherwise specified in infants and toddlers. It consists of 62 items that are read to a parent/caretaker. The parent/caretaker is instructed to rate each item by comparing the child to other children his/her age with the following ratings: 0=not different; no impairment, 1=somewhat different; mild impairment, and 2=very different; severe impairment. The second and third component assess symptoms of emotional difficulties and challenging behaviors that commonly occur with ASD, respectively. The fourth component provides supplemental information related to child's response to name, interest in peers or others, eye contact, pretend play, and engagement in reciprocal play. However, information on the fourth component is purely based on the information obtained from the first three Parts.

The Modified Checklist for Autism in Toddlers (M-CHAT) including the (M-CHAT-Revised with Follow-Up [M-CHAT-R/F]), has a positive predictive value of 48 percent in diverse populations of children ages 16 to 30 months. (Robins 2009)

The Childhood Autism Rating Scale Second edition (CARS2, 2010) is a 15-item observation and parent interview measure that quantifies the severity of behaviors associated with autism. Items are rated on a scale from 1 (‘normal’) to 4 (‘severely abnormal’). Total scores ≥30 strongly suggest the presence of autism.

The First Year Inventory (FYI) is a 63-item parent report questionnaire designed to assess ASD risk in 12-month-old children. It consists of social-communication and sensory-regulatory domains that sum to form a total risk score. (Baranek 2003).

For clinical drug trials for autism, outcome measures commonly used include the Clinical Global Impressions Scale, the Social Responsiveness Scale (Constantino and Gruber 2005), including a preschool version (Stickley 2017), the Autism Behavior Rating Scale, the CARS the Pervasive Developmental Disorder Behavior Inventory (PDDBI, Cohen 2005), the Vineland Adaptive Behavior Scales-II (Sparrow 2005), and the Aberrant Behavior Checklist (Marteleto and Pedromonico 2005).

### Dosing Regimens

In accordance with the treatment methods of the present invention, a therapeutically effective amount of a ROCK inhibitor or a pharmaceutically acceptable salt thereof for administration one or more times a day may comprise from about 10 mg to about 200 mg. The lowest therapeutically effective amount may be determined empirically as the minimum dose that alleviates one or more sign or symptom in a patient treatable according to the invention. The highest therapeutically effective dose may be determined empirically as the highest dose that remains effective in alleviating one or more sign or symptom but does not induce an unacceptable level or adverse events. Fasudil hydrochloride hemihydrate, for example, is suitably administered in a daily amount of about 10 mg to about 200 mg, about 10 mg to about 150 mg, about 10 mg to about 100 mg, about 10 mg to about 70 mg, about 10 mg to about 50 mg. Exemplary total daily dosing with fasudil is from 0.5 to 3 mg/kg of body weight. Preferred daily dosing with fasudil is from 1 to 2 mg/kg of body weight and most preferred daily dosing is from 1.2 to 1.8 mg·kg of body weight. Fasudil is preferably administered as an immediate-release formulation in equal portions two or three times per day. Based on ROCK inhibitory activity, one skilled in the art can readily extrapolate the provided dosing ranges for fasudil to other ROCK inhibitors.

The treatment methods of the present invention, while contemplating various routes of administration, are particularly suited to oral administration. While pills, tablets, capsules and other conventional solid dosage forms are contemplated, especially for use in the pediatric population oral liquids, mini tablets, powders and granules are preferred. In some embodiments, the rho kinase inhibitor can be mixed with food or drink to obtain the appropriate dose. It may be mixed with infant formula, breast milk, baby food, juices or milk, for example.

Another embodiment involves the treatment with 0.5-3 mg/kg of fasudil hydrochloride hemihydrate once per day in an extended release dosage form. Treatment with an extended release total daily dose of 1-2 mg/kg fasudil hydrochloride hemihydrate once per day is preferred.

Methods of administering compositions according to the invention would generally be continued for at least one day. Some preferred methods treat for up to 30 days or up to 60 days or even up to 90 days or even more. Treatment for more than 60 days is preferred and treatment for at least 6 months is particularly preferred. The precise duration of treatment will depend on the patient's condition and response to treatment. Most preferred methods contemplate that treatment begins after the onset or appearance of symptoms.

### Results

The inventive methods result in improvements in neurodevelopmental deficits and their symptoms.

The efficacy of the inventive treatment methods may be assessed in a number of ways. Generally speaking, the inventive methods will result in improvements on any assessments used to identify neurodevelopmental deficits. These scales generally assess capabilities relative to a normal population, with treatable patients exhibiting neurodevelopmental deficits or delays relative to peers of the same age, and so improvements would include normalization or a decrease in the rate of decline. Useful scales include the HNNE, the HINE, the Bayley Scales of Infant and Toddler Development (especially the cognitive scale), the ASQ, the SINDA, the Picciolini neurofunctional assessment, the TINE and the ATNAT. Improvements are preferably see on the Bayley Mental Development Index/Cognition scale and/or the Wechsler Intelligence Scales for Children (WISC). While the Bayley is preferred for younger patients, the WISC is most useful in children aged 6 to 16. The WISC, now in its fifth edition, is an individually administered intelligence test that takes 45-65 minutes to administer. It generates a Full Scale IQ that represents a child's general intellectual ability. It also provides five primary index scores: Verbal Comprehension Index, Visual Spatial Index, Fluid Reasoning Index, Working Memory Index, and Processing Speed Index.

The methods of the invention are considered to be disease modifying, such that they will result in improvements in all related signs and symptoms. Such improvements may be absolute, in that a treated patient will actually show an improvement over time relative to a previous measurement, such as a baseline measurement. Improvements are more typically measured relative to control patients. Control patients may be historical and/or based on the known natural history of similarly-situated patients, or they may be controls in the sense that they receive placebo or simply standard of care in these same clinical trial. Comparison to controls is especially instructive as it is unlikely that the course of the disease will be fully reversed and so results are measure in terms of decreased deterioration relative to controls/expectations.

Improvements resulting from the inventive methods will generally be at least 10%; 15%; 20%; 25%; 30%; 35%; 40%; 45% or 50%, absolute or in comparison to a control. In another embodiment, improvements resulting from the inventive methods will be at least 50% or more, absolute or in comparison to a control. In preferred embodiments, improvements resulting from the inventive methods will be at least 75%, absolute or relative to a control. Patients treated according to the invention are also expected to show improvements in one or more of the following: inappropriate emotions, learning ability, self-control, motor control, forgetfulness, slowing of thought processes, mild intellectual impairment, apathy, inertia, depression, irritability, loss of recall ability, and the inability to manipulate knowledge, mood disorders, repetitive behavior, compulsive behavior, defects in executive function, deficits in speed, deficits in attention, deficits in planning, deficits in monitoring, deficits in memory tasks, aphasia, apraxia, amnesia, recall abnormality, deficits in encoding information, deficits in memory consolidation, lack of spontaneity, perseveration, and/or deficits in spontaneous recall.

In one embodiment, treatment of an ASD patient improves behavioral and/or quality of life measures such as anxiety, distress, hypersensitivity, sleep problems, food-related behaviors, happiness, aggression, relationships with siblings, awareness of danger, and parent stress.

### Combination Therapy

Treatment of neurological disorders with fasudil can include combination therapy with agents such as anti-epileptic or anti-convulsant agents such as valproate or vigabatrin, sleep aids including melatonin, agents that treat symptoms, such as risperidone, aripiprazole, selective serotonin-reuptake inhibitors, anti-anxiety agents, anti-psychotics, simulants and agents for motor symptoms; and experimental or therapeutic drugs including GABA-acting drugs such as riluzole, arbaclofen, intranasal vasopres sin, balovaptan, minocycline, memantine, metformin, esomeprazole, cannabis and suramin.

### LIST OF REFERENCES

Allegra M et al., Pharmacological rescue of adult hippocampal neurogenesis in a mouse modeol of X-linked intellectual disability. Neurobiology of Disease. 2017; 100: 75-86. https://europepmc.org/article/pmc/pmc5346071 #free-full-text
Baranek G T, Watson L R, Crais E, Reznick S. First-Year Inventory (FYI) 2.0. University of North Carolina; Chapel Hill: 2003.
Busti et al., ROCK/PKA Inhibition Rescues Hippocampal Hyperexcitability and GABAergic Neuron Alterations in a Oligophrenin-1 Knock-Out Mouse Model of X-Linked Intellectual Disability. J. Neurosci. 2020; 40(13): 2776-2788.
Chen M, Liu A, Ouyang Y, Huang Y, Chao X, Pi R. 2013. Fasudil and its analogs: a new powerful weapon in the long war against central nervous system disorders? Expert Opin Investig Drugs. 22:537-50.
Cohen I L, Sudhalter V. PDDBI Parent Rating Form. Lutz, F L: Psychological Assessment Resources Inc; 2005.
Constantino J N, Gruber C P (2005). Social Responsiveness Scale (SRS). Los Angeles: Western Psychological Services.
Feng Y, LoGrasso P, Defert O, Li R, Rho Kinase (ROCK) Inhibitors and Their Therapeutic Potential. J Med Chem. 2016; 59*6): 2269-2300.
Hadders-Algra et al., The assessment of minor neurological dysfunction in infancy using the Touwen Infant Neurological Examination: strengths and limitations. Developmental Medicine & Child Neurology. 2009; 52(1): 87-92).
Jacobs M, Hayakawa K, Swenson L, Bellon S, Fleming M, Taslimi P, Doran J, The structure of dimeric ROCK I reveals the mechanism for ligand selectivity. J Biol Chem. 2006; 281(1): 260-68.
Marteleto M R, Pedromonico M R. Validity of Autism Behavior Checklist (ABC): preliminary study. Rev Bras Psiquiatr. 2005; 27:295-301.
Meziane et al., Fasudil treatment in adult reverses behavioural changes and brain ventricular enlargement in Oligophrenin-1 mouse model of intellectual disability. Human Molecular Genetics, 2016; 25(11): 2314-2323.
Nakagawa O, Fukisawa K, Ishizaki T, Saito Y, Nakao K, Narumiya S, ROCK-I and ROCK-II, two isoforms of Rho-associated coiled-coil forming protein serine/threonine kinase in mice. IHEBS Lett. 1996 Aug. 26; 392(2):189-93.
Yamaguchi H, Miwa Y, Kasa M, Kitano K, Amano M, Kaibuchi K, Hakoshima T, Structural basis for induced-fit binding of Rho-kinase to the inhibitor Y-27632. J Biochem. 2006 September; 140(3):305-11.
Uehata M, Ishizaki T, Satoh H, Ono T, Kawahara T, Morishita T, Tamakawa H, Yamagami K, inui J, Maekawa M, Narumiya S, Calcium sensitization of smooth muscle mediated by a Rho-associated protein kinase in hypertension. Nature. 1997 Oct. 30; 389(6654):990-4.
Shibuya M, Asano T, Sasaki Y. 2001. Effect of Fasudil HCl, a protein kinase inhibitor, on cerebral vasospasm. Acta Neurochir Suppl. 77:201-4.
Matson J L, Wilkins J, Sharp B, et al. Sensitivity and specificity of the Baby and Infant Screen for Children with Autism Traits (BISCUIT): Validity and cutoff scores for autism and PDD-NOS in toddlers. Research in Autism Spectrum Disorders. 2009; 3(4):924-30.
Robins, Fein and Barton, Modified Checklist for Autism in Toddlers, Revised, with Follow Up. 2009 Self-published
Robins D L, Casagrande K, Barton M, et al. Validation of the Modified Checklist for Autism in Toddlers, Revised With Follow-up (M-CHAT-R/F). Pediatrics 2013 December 23PMID: 24366990.
Sparrow S, Cicchetti D, Balla D. Vineland Adaptive Behavior Scales (Vineland II): Parent/Caregiver Rating Form. 2nd ed. Minneapolis, MN: NCS Pearson Inc, 2005.
Stickley A, Tachibana Y, Hashimoto K, Haraguchi H, Miyake A, Morokuma S, et al. Assessment of autistic traits in children aged 2 to 4½ years with the preschool version of the Social Responsiveness Scale (SRS-P): Findings from Japan. Autism Res. 2017
Touwen B C L. Neurological development in infancy. Clinics in Dev. Med. 1976; 58. London: Mac Keith Press and limitations. Developmental Medicine & Child Neurology 2010, 52: 87-9

What is claimed is:

1. A method of treating a patient having a neurodevelopmental disorder and having a copy number variation in 16p11.2, comprising orally treating said patient with fasudil at a daily dose of between 10 mg and 200 mg.

2. A method of treating a patient having a neurodevelopmental disorder with and having a copy number variation in 16p11.2, comprising orally treating said patient with fasudil at a daily dose of between 0.5 to 3 mg/kg.

3. The method according to claim 2, wherein the fasudil is administered as an extended release formulation.

4. The method according to claim 1, wherein said copy number variation is a deletion.

5. The method according to claim 1, wherein said copy number variation is a duplication.

6. The method of claim 2, wherein said copy number variation is a deletion.

7. The method of claim 2, wherein said copy number variation is a duplication.

8. The method of claim 1, wherein the neurodevelopmental disorder is Autism Spectrum Disorder.

9. The method of claim 2, wherein the neurodevelopmental disorder is Autism Spectrum Disorder.

10. The method of claim 8, wherein the copy number variation is a deletion.

11. The method of claim 9, wherein the copy number variation is a deletion.

12. The method of claim 1, wherein the patient is a child having a learning disability.

13. A method of treating a patient having Autism Spectrum Disorder comprising orally treating said patient with fasudil at a daily dose of between 10 mg and 200 mg.

14. The method of claim 13, wherein the daily dose is between 0.5 to 3 mg/kg.

15. The method of claim 14, wherein the fasudil is administered in an extended release formulation.

16. The method of claim 13, wherein the patient has a learning disability.
